(19) United States
(12) Patent Application Publication
(10) Pub. No.: US 2016/0326601 A1
(43) Pub. Date: Nov. 10, 2016

(54) METHOD FOR DETECTING A CHICKEN BEARD TRAIT

(71) Applicant: CHINA AGRICULTURE UNIVERSITY, Beijing (CN)

(72) Inventors: Xiaoxiang HU, Beijing (CN); Xiaorong GU, Beijing (CN); Ying GUO, Beijing (CN); Zheya SHENG, Beijing (CN); Yanqiang WANG, Beijing (CN); Hao QU, Beijing (CN); Ning LI, Beijing (CN)

(21) Appl. No.: 14/913,343

(22) PCT Filed: Oct. 29, 2013
(86) PCT No.: PCT/CN2013/086097

§ 371 Date: Feb. 19, 2016

(30) Foreign Application Priority Data
Aug. 21, 2013 (CN) 201310367724.5

(51) Int. Cl. C12Q 1/68

(52) U.S. Cl. CPC C12Q 1/6888; C12Q 2600/124; C12Q 2600/156

(57) ABSTRACT

The present invention provides a method for detecting a chicken Beard trait. Copy number variations of three DNA fragments were found to be present in the chromosomes of bearded chicken number 27, in the physical locations 1702269-1721521 bp, 4470331-4503417 bp, 3578409-3592890 bp; 3 molecular markers were determined, and 3 pairs of primers were designed for said molecular markers; the method of PCR is used for testing the chicken genome to be tested for the described 3 molecular markers; if the three target fragments of the PCR amplification reaction are 3200 bp, 501 bp, and 411 bp, then the detection result is positive; otherwise, the result is negative.

### TECHNICAL FIELD

The present invention relates to the technical field of molecular biology, specifically to molecular markers for Beard trait in chickens and a method for detecting the Beard trait in chickens.

### BACKGROUND

Long-term evolution and selection by domestication have enriched the diversity of agricultural animal genetic resources. Recent studies have found that genomic structural variations are the genetic basis of many important traits during livestock and poultry domestication. For example, complex structure rearrangements occurring on chromosome 20 of chicken will change the expression of gene EDN3, eventually leading to the deposition of melanin in the dermis; and the duplication of a 3.2 kb region in intron 1 of gene SOX5 located on chromosome 1 of chicken changes the expression of gene SOX5 within 6-12 days of development, thereby resulting in the generation of a pea-comb phenotype. The genomic structural variations may affect gene expression through multiple mechanisms and thus studies on the genomic structural variations may help us understand and analyze the mechanisms underlying the formation of related traits.

Genomic structural variations include four types, i.e. deletion, duplication, inversion and translocation. Currently, technologies for detecting genomic structural variations are mainly based on high-density SNP genotyping chips, comparative genomic hybridization chips, high-throughput sequencing technology and the like. With the continuous development of high-throughput sequencing technology and the continual decreasing of cost, sequencing has become an important means in current biological researches. The information of breakpoints and rearrangements caused by the structural variations may be obtained by re-sequencing analysis of the target region with structural variations, and then the structural variations may be detected by conventional molecular biological techniques such as PCR. For detection, it is only needed to design amplification primers for both sides of the breakpoints generated by the structural variations, and the presence of which may be detected by agarose gel electrophoresis using simple PCR reaction.

The Beard trait in chickens is one of the phenotypic traits that have been early studied in the poultry, and yet there has been no report on the definite chromosomal location that affects the Beard trait and method for detection for hundreds of years. If a traditional method is used to perform selection for the Beard trait, a lot of manpower, material and financial resources will be consumed. A method for rapid detection of the Beard trait established by molecular biological means and thus used for the marker-assisted selection will help improve breeding efficiency.

### DISCLOSURE OF THE INVENTION

The purpose of the present invention is to provide a method for identifying Beard trait in chickens.

In the present invention, the objects of study are F2 hybrid resource population established by China Agriculture University in cooperation with Institute of Animal Science, Guangdong Academy of Agricultural Science, using Huiyang Bearded Chicken and High quality of chicken line A (A03) as parents. The Beard trait of F0, F1 and F2 individuals are recorded and the loci that affect the Beard trait in chickens are mapped on GGA27 by using genome-wide SNP linkage analysis. Re-sequencing analysis is performed for F0 samples, and the structural variations affecting the Beard trait in chickens are obtained. It is found in the present invention that three DNA fragment duplications (copy number variation, CNV) are present on GGA27 of the Bearded Chicken and located at the following physical locations in chicken genome, ICGSC Gallus_gallus-4.0 version, respectively: 1702269-1721521 bp (CNV1), 4470331-4503417 bp (CNV2) and 3578409-3592890 bp (CNV3). The three fragments are linked smoothly and inserted into original CNV1 loci with a positional relationship shown in FIG. 1. The method for identifying the Beard trait in chickens according to the present invention comprises: designing primers of linker fragments between different CNV fragments to perform PCR detection and determining whether or not a chicken possesses the Beard gene according to the detection results.

In the first aspect, the present invention provides a combination of molecular markers for detecting a Beard trait in chickens, wherein the molecular markers are CNV1, CNV2 and CNV3, which are respectively located at the following physical locations of GGA27: 1702269-1721521 bp, 4470331-4503417 bp and 3578409-3592890 bp; the gene sequence of a linker fragment between CNV1 and CNV2 is shown in SEQ ID No.7, the gene sequence of a linker fragment between CNV2 and CNV3 is shown in SEQ ID No.8, and the gene sequence of a linker fragment between CNV3 and CNV1 is shown in SEQ ID No.9.

In the second aspect, the present invention provides a combination of primers for detecting the combination of molecular markers, wherein the primers are designed for the linker fragments between the different CNV fragments, whose nucleotide sequences are as follows:

| | |
|---|---|
| CNV1-2F | TTTGTTGTCTCCTTGCATCATT |
| CNV1-2R | CCATGTCAGCACAGTAACGATT |
| CNV2-3F | ATGTTGGTTGTGTGCCAAGTAG |
| CNV2-3R | TTTCCCCTCGTCTGCTTTATTA |
| CNV3-1F | ACAAGTCAGAGAGGCAATCGAC; |
| and | |
| CNV3-1R | CCATACAGCCTCAGGTAAGGAC. |

In the third aspect, the present invention provides a kit for detecting a Beard trait in chickens, comprising 3 pairs of primers designed respectively for the three molecular markers described above.

The method according to the present invention comprises the steps of:

(1) Performing PCR reactions using genomic DNA of a chicken to be tested as a template with the following three primer pairs, respectively:

| | |
|---|---|
| CNV1-2F | TTTGTTGTCTCCTTGCATCATT |
| CNV1-2R | CCATGTCAGCACAGTAACGATT |
| CNV2-3F | ATGTTGGTTGTGTGCCAAGTAG |
| CNV2-3R | TTTCCCCTCGTCTGCTTTATTA |
| CNV3-1F | ACAAGTCAGAGAGGCAATCGAC |
| CNV3-1R | CCATACAGCCTCAGGTAAGGAC; |

and

(2) Detecting the amplification products of three PCR reactions on agarose gel, and if target fragments of the PCR amplification reactions for CNV1, CNV2 and CNV3 primers are 3200 bp, 501 bp and 411 bp, respectively, the detection result is positive, otherwise the result is negative.

The reaction system for PCR amplification for primer pairs CNV3-1F and CNV3-1R, and CNV2-3F and CNV2-3R in step (1) in the method according to the present invention is as follows:

In the case of the total system is of 25 μ1: genomic DNA: 50 ng, 1× PCR Buffer, dNTP 4 mM, each of upstream and downstream primers for 10 pmol, Taq DNA polymerase (Kangwei™) 1.25 U, and adding water into the reaction system to 25 μl.

The reaction system for PCR amplification for primer pair CNV1-2F and CNV1-2R is as follows: genomic DNA: 50 ng, 1× PCR Buffer, dNTP 4 mM, each of upstream and downstream primers for 10 pmol, Long Amp® Taq DNA polymerase (NEB) 1.25 U, and adding water into the reaction system to 25 μl.

The PCR reaction conditions for primer pairs CNV3-1F and CNV3-1R, and CNV2-3F and CNV2-3R in step (1) in the method according to the present invention are as follows: predenaturation at 94° C. for 5 min; 35 cycles: denaturation at 94° C. for 30 sec, annealing at 60° C. for 30 sec, and extension at 72° C. for 20 sec; final extension at 72° C. for 7 min and storage at 12° C.

The PCR reaction condition for primer pair CNV1-2F and CNV1-2R is as follows: predenaturation at 94° C. for 3 min; 35 cycles: denaturation at 94° C. for 10 sec, annealing at 57° C. for 30 sec, and extension at 65° C. for 4 sec; final extension at 65° C. for 7 min and storage at 12° C.

The present invention provides the use of the method and the combination of molecular markers described above in chicken breeding.

The present invention further provides the use of the combination of the primers described above in the detection of a Beard trait in chickens or the preparation of a kit for detecting the Beard trait in chickens.

The present invention provides 3 molecular markers for identifying a Beard trait for the first time. Using the above mentioned molecular markers, a total of 3 pairs of specific primers are designed. PCR detection is performed for the genomic DNA of a chicken to be tested respectively and whether or not the chicken to be tested possesses a Beard trait will be determined according to the detection results. The method according to the present invention may achieve a fast screening of chicken individuals possessing a Beard trait and accelerate the breeding of chickens with the Beard trait, thereby substantially improving the efficiency of breeding and breed conversation. The detection method according to the present invention is simple in operation and has low cost and high accuracy.

### BRIEF DESCRIPTION OF THE DRAWINGS

FIG. 1 shows the logarithm value (Log2 Ratio) of sequencing depth ratio from the re-sequencing of a Bearded Chicken and High quality of chicken line A (A03) provided in the present invention. The horizontal axis is physical map sequence location of GGA27. Three CNVs are present on GGA27 possessing the Beard trait, wherein the interval for CNV1 is chr27:1702269-1721521 bp; the interval for CNV2 is chr27:4470331-4503417 bp; and the interval for CNV3 is chr27:3578409-3592890 bp. After replication, CNV1, CNV2 and CNV3 are inserted into the downstream of the original position of CNV1 and they are linked in a way as shown in FIG. 1.

FIG. 2 shows the results of agarose gel electrophoresis detection after amplification with the primes CNV1-2F and CNV1-2R provided in the present invention. The sizes of the target fragment and the marker are 3.2 kb and 1 kb, respectively. Lanes 13-24 are Beijing Fatty Chicken (bearded) with amplified bands; and Lanes 1-12 are High quality of chicken line A (non-bearded) without amplified bands.

FIG. 3 shows the results of agarose gel electrophoresis detection after amplification with the primes CNV2-3F and CNV2-3R provided in the present invention. The sizes of the target fragment and the marker are 501 bp and 100 bp, respectively. Lanes 13-24 are Beijing Fatty Chicken (bearded) with amplified bands; and Lanes 1-12 are High quality of chicken line A (non-bearded) without amplified bands.

FIG. 4 shows the results of agarose gel electrophoresis detection after amplification with the primes CNV3-1F and CNV3-1R provided by the present invention. The sizes of the target fragment and the marker are 411 bp and 100 bp, respectively. Lanes 13-24 are Beijing Fatty Chicken (bearded) with amplified bands; and Lanes 1-12 are High quality of chicken line A (non-bearded) without amplified bands.

FIG. 5 is the MANHATTAN plot resulted from genome-wide association studies of the Beard trait in chickens. FIG. 5a shows that only the markers on GGA27 are genome-level significant (the horizontal axis is the number of different chromosomes, and the vertical axis is base-10 logarithm value of p value). FIG. 5b shows the association signal of markers on GGA27 (the horizontal axis is the physical location of GGA27, and the vertical axis is base-10 logarithm value of p value). A dotted line represents 5% genome-level significance corrected by Bonferroni.

### SPECIFIC MODE FOR CARRYING OUT THE PRESENT INVENTION

The present invention will be better understood by the following examples, but is not limited thereto. Unless otherwise stated, the experimental methods in the examples below are conventional methods. Unless otherwise stated, the experimental materials used in the examples below may be obtained from general stores selling biochemical reagents.

### EXAMPLE 1

### Determination of Three Copy Number Variation Intervals on GGA27 1. QTL Mapping of a Beard Trait

In the present invention, the objects of study are F2 hybrid resource population established by China Agriculture University in cooperation with Institute of Animal Science, Guangdong Academy of Agricultural Science, using Huiyang Bearded Chicken and High quality of chicken line A (A03) as parents. The Beard traits of individuals of F0, F1 and F2 generations are recorded. Genotyping is performed using a chicken 60 K SNP chip of Illumina and the information of genome-wide SNP markers with a mean size of about 42 K in 588 individuals is obtained through data quality control (detection rate of sample >90%; detection rate of SNP >90%; minor allele frequency >0.05; and genetic error rate <0.05). It is found that all of SNPs reaching a genome-level significance of 5% corrected by Bonferroni (P<1.19E-6) are located on GGA27 (as shown in FIG. 5a and FIG. 5b), but the interval for association signals is larger by the mixed model and regression analysis using the GenABEL software.

A linkage map of GGA27 is established using CRI-MAP software. A minimum haplotype (1.70-1.72 Mb) of 22 Kb that affects the Beard trait in chickens is identified by genetic segregation analysis and re-sequencing analysis of recombinants within intervals.

2. Copy Number Variations and the Beard Trait

In the present invention, the information of genome-wide copy number variations from 14 chicken breeds (only Bearded Chicken has the Beard trait) is analyzed by using a custom-made Agilent 2X400K comparative genomic hybridization (CGH) chip with the genome of a Dehong chicken as reference. CNVs comprising 5 continuous probes can be identified as confident CNV regions, and thereby 3 CNV regions specific for Bearded Chicken breeds are detected (see Table 1; CNVR1, CNVR2 and CNVR4). CNVR1 is completely overlapped with the haplotype of the Beard trait and thus it can be inferred that the copy number variations may be mutations resulting in the Beard trait phenotype.

TABLE 1
Information of CNVs Specific for Bearded Chicken Breeds

| | | Initiation | Termination | | Number | Number of |
|---|---|---|---|---|---|---|
| No. | Chromosome | Site | Site | Size | of Probes | Acquisition |
| CNVR1 | 27 | 1,702,671 | 1,720,386 | 17,715 | 10 | 2 |
| CNVR2 | 27 | 4,471,823 | 4,503,373 | 31,550 | 17 | 2 |
| CNVR3 | 27 | 3,581,044 | 3,583,446 | 2,402 | 2 | 2 |
| CNVR4 | 2 | 141,819,961 | 141,834,442 | 14,481 | 7 | 2 |

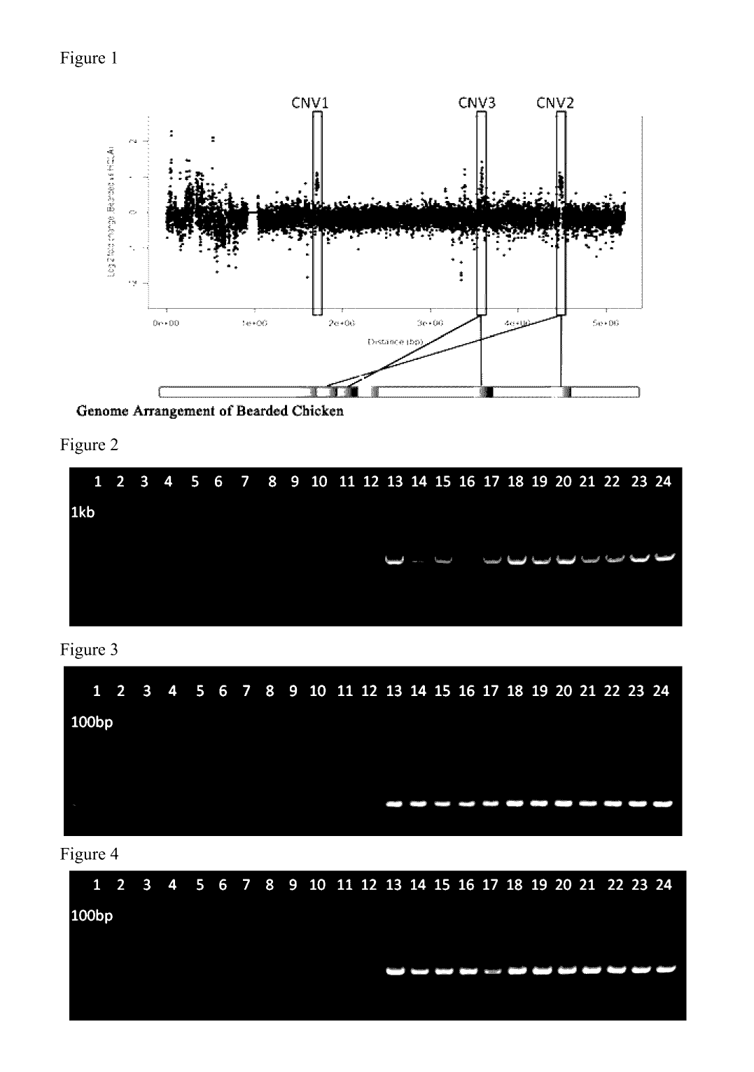

The genome-wide DNA re-sequencing analysis was performed for F0 samples of the resource family, and a study on genomic structural variations was conducted by the read depth analytical method, as shown in FIG. 1. The GGA27 was plotted using base-2 logarithm values of the read depth ratio of Bearded Chicken to High quality of chicken line A (log₂ ratio). For 1.7 Mb, 3.5 Mb and 4.4 Mb intervals, the log₂ratio reaches 1, which indicates that the read depth ratio of Bearded Chicken to High quality of chicken line A is 2 and all the three intervals are multi-copy structural variations, that is, a normal genome has 2 haplotypes and the Bearded Chicken has 4 haplotypes. The results of CNVR1 and CNVR2 are consistent with that of CGH. Since CNVR3 fragment is relatively small, it is not included in the initial statistics.

Three breakpoints of structural variation are identified by the partially-mapped read information, which are 1702269-1721521 bp (CNVR1), 4470331-4503417 bp (CNVR2) and 3578409-3592890 bp (CNVR3), respectively. The gene sequences of a linker fragment between CNV1 and CNV2, between CNV2 and CNV3, and between CNV3 and CNV1 are shown as SEQ ID No.7, SEQ ID No.8 and SEQ ID No.9, respectively. Meanwhile, the new copy fragments of the three CNVs are linked smoothly and inserted into the original CNVR1 loci with a positional relationship shown in FIG. 1, rather than exist independently.

### EXAMPLE 2

### Designing of Primers for the Three CNV Molecular Markers

The primers for linker fragments between different CNV fragments viz. 1702269-1721521 bp (CNV1), 4470331-4503417 bp (CNV2) and 3578409-3592890 bp (CNV3) determined in Example 1 are designed for PCR detection according to the present invention. The primer sequences are shown in Table 2.

TABLE 2
The nucleotide sequences of the
three primer pairs

| | |
|---|---|
| CNV1-2F | TTTGTTGTCTCCTTGCATCATT |
| CNV1-2R | CCATGTCAGCACAGTAACGATT |
| CNV2-3F | ATGTTGGTTGTGTGCCAAGTAG |
| CNV2-3R | TTTCCCCTCGTCTGCTTTATTA |
| CNV3-1F | ACAAGTCAGAGAGGCAATCGAC |
| CNV3-1R | CCATACAGCCTCAGGTAAGGAC |

### EXAMPLE 3

### Establishment of a Method for Detecting Beard Trait in Chickens

1. Experimental Materials

The tested Beijing Fatty Chickens are from the Institute of Animal Sciences, Chinese Academy of Agricultural Sciences; Xiangdong Chickens are from Wuping breed conversation farm for Xiangdong Chicken, Longyan, Fujian, China; the hybrid resource population of Huiyang Bearded Chicken and High quality of chicken line A (A03) is from Institute of Animal Science, Guangdong Academy of Agricultural Science; and other breeds are from Jiangsu Poultry Institute, Chinese Academy of Agricultural Science.

2. Extraction of Genomic DNA

The blood samples were taken from the wing vein of chickens, followed by anticoagulation treatment and lysis, then digested overnight with proteinase K, extracted with phenol and chloroform, dissolved in TE and stored at −20° C.

3. PCR Amplification

PCR reaction was performed for the genome of the samples with three primer pairs in Example 2, respectively.

The reaction system for PCR amplification for primer pairs CNV3-1F and CNV3-1R, and CNV2-3F and CNV2-3R is as follows: genomic DNA: 50 ng, 1× PCR Buffer, dNTP 4 mM, forward primer 10 pmol, reverse primer 10 pmol, Taq DNA polymerase (Kangwei™) 1.25 U, and adding water into the reaction system to 25 μl. The reaction system for PCR amplification for primer pair CNV1-2F and CNV1-2R is as follows: genomic DNA: 50 ng, 1× PCR Buffer, dNTP 4 mM, forward primer 10 pmol, reverse primer 10 pmol, Long Amp® Taq DNA polymerase (NEB) 1.25 U, and adding water into the reaction system to 25 μl.

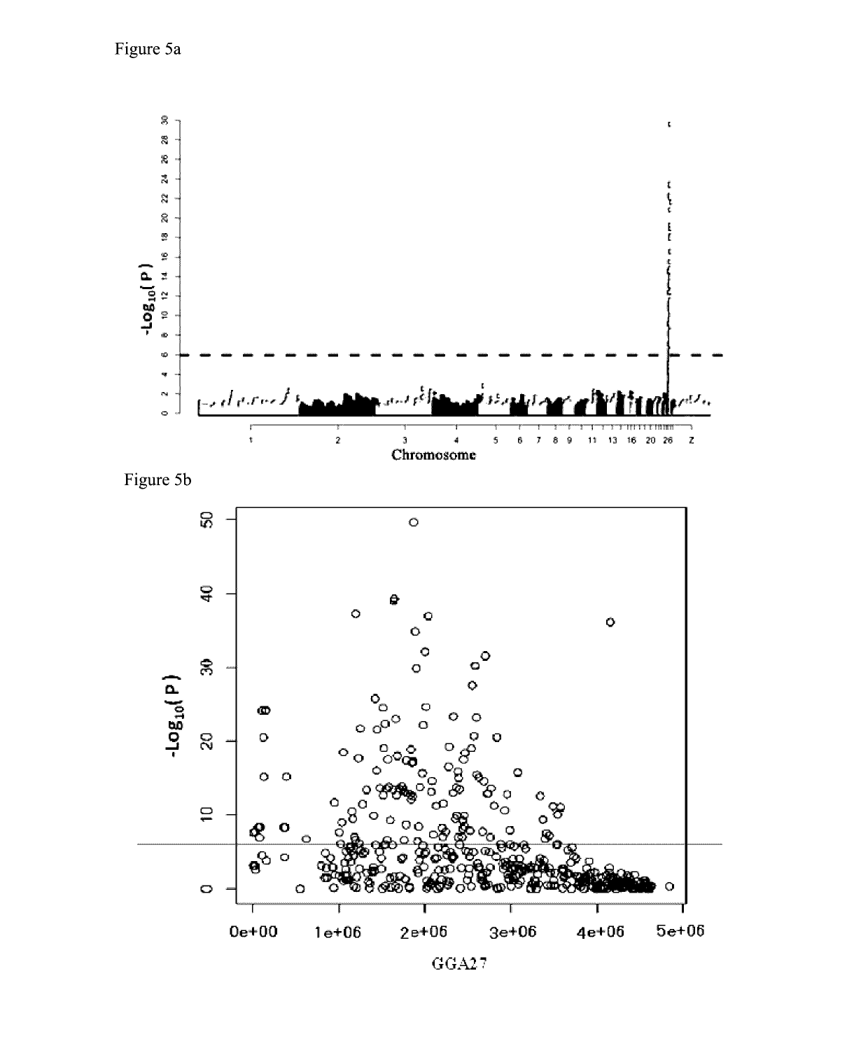

The PCR reaction conditions for primer pairs CNV3-1F and CNV3-1R, and CNV2-3F and CNV2-3R are as follows: pre-denaturation at 94° C. for 5 min; 35 cycles: denaturation at 94° C. for 30 sec, annealing at 60° C. for 30 sec, and extension at 72° C. for 20 sec; final extension at 72° C. for 7 min and storage at 12° C. The PCR reaction condition for primer pair CNV1-2F and CNV1-2R is as follows: pre-denaturation at 94° C. for 3 min; 35 cycles: denaturation at 94° C. for 10 sec, annealing at 57° C. for 30 sec, and extension at 65° C. for 4 sec; final extension at 65° C. for 7 min and storage at 12° C. Amplification products are detected on 2% agarose gel. The amplification results are shown in FIGS. 2, 3 and 4.

4. Analysis of Results

For individuals possessing the Beard trait, target bands can be obtained by the amplification with three primer pairs according to the present invention. For individuals not possessing the Beard trait, the targets bands cannot be obtained by amplification. In the tested 524 individuals, 248 individuals were identified to be bearded, and 276 individuals were identified to be non-Bearded. The results are shown in Table 3.

TABLE 3
Results of detection for Beard trait

| | Number of | | Non- |
|---|---|---|---|
| Breed | Individuals | Bearded | Bearded |
| High quality of chicken line A | 18 | — | 18 |
| Huiyang Bearded Chicken | 18 | 18 | — |
| F1 family of Huiyang Bearded and High | 36 | 36 | — |
| quality of chicken line A | | | |
| F2 family of Huiyang Bearded and High | 89 | 62 | 27 |
| quality of chicken line A | | | |
| F5 family of Huiyang Bearded and High | 189 | 95 | 94 |
| quality of chicken line A | | | |
| Wenchang Chicken | 6 | — | 6 |
| Qingyuan Partridge Chicken | 6 | — | 6 |
| Beijing Fatty Chicken | 23 | 11 | 12 |
| Xiangdong Chicken | 20 | 20 | — |
| White-ear Yellow Chicken | 6 | — | 6 |
| Wahui Chicken | 6 | — | 6 |
| Chahua Chicken | 6 | — | 6 |
| Henan Gamecock | 6 | — | 6 |
| Chongren Partridge Chicken | 6 | — | 6 |
| Langshan Chicken | 6 | — | 6 |
| Bian Chicken | 6 | — | 6 |
| Luyuan Chicken | 6 | — | 6 |
| Tibetan Chicken | 6 | — | 6 |
| Gushi Chicken | 6 | — | 6 |
| Dagu Chicken | 6 | — | 6 |
| Youxi Partridge Chicken | 6 | — | 6 |
| Anak Chicken | 6 | — | 6 |
| Recessive White Chicken | 6 | — | 6 |
| Shouguang Chicken | 6 | — | 6 |
| Shiqiza Chicken | 6 | — | 6 |
| Dwarf Yellow Chicken | 6 | — | 6 |
| Red Jungle Fowl | 6 | — | 6 |
| Ladle Chicken | 5 | — | 5 |
| Total | 518 | 242 | 276 |

The above described identification results of 524 individuals indicate that the Beard trait in chickens is fully associated with 3 CNVs on GGA27, and thus the presence of the Beard trait may be determined by judging structural variation events on GGA27. Such method for detecting the Beard trait may be used in breeding practice, which overcomes the time- and labor-consuming defects of conventional breeding methods and accelerates the selection of chickens possessing the Beard trait.

In principle, the conclusion whether or not a Beard trait is present may be drawn by using any one of the three primer pairs according to the present invention, but the reliability of the results cannot be assured. Therefore, in the present invention, it is required to perform amplification with 3 primer pairs respectively and to determine whether or not the chicken breed to be tested possesses the Beard trait based on the results of the three PCR detections so as to assure the correctness of the result of the determination.

Although the present invention and embodiments thereof have been described above in detail, it should be noted that, those skilled in the art may make modifications to corresponding conditions without departing from the scope and spirit of the invention and such modifications should also be regarded as falling into the protection scope of the present invention.

### INDUSTRIAL APPLICABILITY

The present invention provides 3 molecular markers and 3 pairs of specific primers for detecting the same. PCR detection is performed for the genomic DNA of a chicken to be tested respectively and whether or not the chicken to be tested possesses a Beard trait will be determined according to the detection results. The method according to the present invention may achieve a fast screening of chicken individuals possessing the Beard trait and accelerate the breeding of chickens having the Beard trait, thereby substantially improving the efficiency of breeding and breed conversation. This detection method according to the present invention is simple in operation has low cost and high accuracy, and thus it has important economic value and application prospective.

1. A combination of molecular markers for detecting a Beard trait in chickens, characterized in that the molecular markers are CNV1, CNV2 and CNV3, which are respectively located at the following physical locations of GGA27: 1702269-1721521 bp, 4470331-4503417 bp and 3578409-3592890 bp; the gene sequence of a linker fragment between CNV1 and CNV2 is shown in SEQ ID No.7, the gene sequence of a linker fragment between CNV2 and CNV3 is shown in SEQ ID No.8, and the gene sequence of a linker fragment between CNV3 and CNV1 is shown in SEQ ID No.9.

2. Use of the combination of molecular markers of claim 1 in chicken breeding.

3. A combination of primers for detecting the combination of molecular markers of claim 1, characterized in that nucleotide sequences of the primer sequences are as follows:

| | |
|---|---|
| CNV1-2F | TTTGTTGTCTCCTTGCATCATT |
| CNV1-2R | CCATGTCAGCACAGTAACGATT |
| CNV2-3F | ATGTTGGTTGTGTGCCAAGTAG |
| CNV2-3R | TTTCCCCTCGTCTGCTTTATTA |
| CNV3-1F | ACAAGTCAGAGAGGCAATCGAC; |
| and | |
| CNV3-1R | CCATACAGCCTCAGGTAAGGAC. |

4. Use of the combination of primers of claim 3 in the preparation of a kit for detecting a Beard trait in chickens.

5. Use of the combination of primers of claim 3 in chicken breeding.

6. A kit for detecting a Beard trait in chickens, characterized in that the kit comprises the combination of primers of claim 3.

7. A method for detecting a Beard trait in chickens, characterized in that the method comprises the steps of:
(1) performing PCR reactions using genomic DNA of a chicken to be tested as a template with the following three primer pairs, respectively:

| | |
|---|---|
| CNV1-2F | TTTGTTGTCTCCTTGCATCATT |
| CNV1-2R | CCATGTCAGCACAGTAACGATT |
| CNV2-3F | ATGTTGGTTGTGTGCCAAGTAG |
| CNV2-3R | TTTCCCCTCGTCTGCTTTATTA |
| CNV3-1F | ACAAGTCAGAGAGGCAATCGAC |
| CNV3-1R | CCATACAGCCTCAGGTAAGGAC; |

and
(2) detecting the amplification products of the three PCR reactions on agarose gel, and if target fragments of the PCR amplification reactions for CNV1, CNV2 and CNV3 primers are 3200 bp, 501 bp and 411 bp respectively, the result is positive, otherwise the result is negative.

8. The method of claim 7, characterized in that the reaction system for PCR amplification for primer pairs CNV3-1F and CNV3-1R, and CNV2-3F and CNV2-3R in step (1) is as follows:
In the case of the total system is of 25 μl: genomic DNA: 50 ng, 1× PCR Buffer, dNTP 4 mM, each of upstream and downstream primers for 10 pmol, Taq DNA polymerase (Kangwei™) 1.25 U, and adding water into the reaction system to 25 μl; and the reaction system for PCR amplification for primer pair CNV1-2F and CNV1-2R is as follows: genomic DNA: 50 ng, 1× PCR Buffer, dNTP 4 mM, each of upstream and downstream primers for 10 pmol, Long Amp® Taq DNA polymerase (NEB) 1.25 U, and adding water into the reaction system to 25 μl.

9. The method of claim 7, characterized in that the PCR reaction conditions for primer pairs CNV3-1F and CNV3-1R, and CNV2-3F and CNV2-3R in step (1) are as follows: predenaturation at 94° C. for 5min; 35 cycles: denaturation at 94° C. for 30sec, annealing at 60° C. for 30 sec, and extension at 72° C. for 20 sec; final extension at 72° C. for 7min and storage at 12° C.; and
the PCR reaction condition for the primer pair CNV1-2F and CNV1-2R is as follows: predenaturation at 94° C. for 3 min; 35 cycles: denaturation at 94° C. for 10 sec, annealing at 57° C. for 30 sec, and extension at 65° C. for 4 sec; final extension at 65° C. for 7 min and storage at 12° C.

10. Use of the method of claim 7 in chicken breeding.

11. Use of the method of claim 8 in chicken breeding.

12. Use of the method of claim 9 in chicken breeding.


SEQUENCE LISTING (9 sequences)

SEQ ID NO: 1 - DNA, 22 bp, Gallus(red jungle fowl)
tttgttgtctccttgcatcatt

SEQ ID NO: 2 - DNA, 22 bp, Gallus(red jungle fowl)
ccatgtcagcacagtaacgatt

SEQ ID NO: 3 - DNA, 22 bp, Gallus(red jungle fowl)
atgttggttgtgtgccaagtag

SEQ ID NO: 4 - DNA, 22 bp, Gallus(red jungle fowl)
tttcccctcgtctgctttatta

SEQ ID NO: 5 - DNA, 22 bp, Gallus(red jungle fowl)
acaagtcagagaggcaatcgac

SEQ ID NO: 6 - DNA, 22 bp, Gallus(red jungle fowl)
ccatacagcctcaggtaaggac

SEQ ID NO: 7 - DNA, 3140 bp, Gallus(red jungle fowl)
tttgttgtctccttgcatcattgctcggtggttgtgggctttcaggttggctctggagcctccacccctgtgagaagggtttctgatgggctcaggatgggtcgtgctgcctggagcctgggaatggtccccctctgtgcctcccatccccgtgggttggggggcaaaggctgctggataccgcaggacggagcagggacatgcttccagcactcctcagtcaacgtgctctgagagcatcagcttaaggtgctgcttggtgctgagcaccccgggtacagagatgggaacttcagtggcaaggtcccttgcttggccctcaacatcctttccagccccatcccaggcactgagctgccctacggtgctccatcaccacctgccttcccagacactcggtattcaatgagattaaatctgtcttcctgtgtggttttctcccttcatgggacggcactgctgggggtgctgatatcaacacagaccctcctcctctcttcccaaaattaccattgaagggacctggcagcatcgacccctccatatacctgttgtgtttgggcacaagagggcactggaacaccgctgtgatgggatgctttgtggctgcaagcagagctgctgcggccgcaaagtccctctgcacctttccccagcgagcagacatccataggacgacctgcactctgctgcacccccttccatttaaatcacactgctctgggagtcggcagttcctcctgcatgatcacagctgccttccctgccgcttcctatagactggatcgatgggccaaggttaatggcatgagtttcaatagggccaagtgtcgggtcctgcattttggtcacaacaaccccaggcagccctacaggcttggggaggagtgtctggaaagctgcctgatggaaagggaccttggtgtactgttggacagtgggctgaatatgagccagcagtgtgcccaggtggccaagaaggtcaatggcatcctggcttggatcaggaatggtgtggtgagcagcactggggaagtcatcctgccctgtacttggcactggtgaggcctcacctcgagtgctgtgttcagttttgggctcctcggtacagaaaggacatggaggtgctggagaaggtccagagaagggcaacaaggctggggaagggcttggagaatatggcctacgaggagagactgaaggaactggggctgtttggtctggggaagaggaggctgaggggagaccttattgctctcttcaaatccctaacaggtgattgcagtgagattggggtggtctcttctcactgctgacaggacaagatgaggggaaatggccccaagttgcgccaagataagttgaggttggacgttaggaaacgcttctttacagaaagggtggttaagcactgggatgggctccccagggaggtggttgagtcgccatccctggatgtgtttaagagccgtttggatgtggtgctcagagatgtgatttagcagagggctgttagagttggggtactgtggttaggctgcggttggacgtgatgatctttgaggtcctttccaacctgagtgttgaagactgagatggtcttggccagcatgacaccgttgtagggcagctggaagacgacgaagacgatgaccaccgcaatgatcaccttgatggctttattcttctcaaagtttcgggcctggagtaacgttttgataatgatgaggtagcagacagacatgaccaccaggggaactaagaagccaaaaaccatctgggacactttgatgccagtgctgaaagtctgcaagtcactggcaatgatggagcaccggggatggctgtcatagttatttacaccactgtgaaccagctcggggatggagagaacgaaggccaggagccaaatgaggatgcacgtgactttgctaatgaatatcattcggggacggaagcggtgggccgaggcagcctgaacgatggcaaagtacctgtcgatgctgatggacagcaggagcagcatcccgctgaagaagctcattttgcagatgcagtagactgctttgcaggcaaattctccaaaacaccagaacgtggctgcacttgtggcccaaaatgggagggtcagcaggaacaggatgtctgccagggccaggttcagcaagtagatgtctgtcatggtcttcagccgcttgaagtagatgtaggtgagcatcaccagcccatttcccagcagccctgtgaagcagatgagggagtacatggctgggaggaaggcagcacggaaatcacggacctccttcttctcacacatcatctcaaacatgttgtagtcaatggtggtgttggcgtcatagtcgtcggtgacgttgttcccagcacagaactagaacagggaaggaagaaagcccgttgagacaggtactgggggtgcgcggcctctgcccctcagtgggtggtttccagtgggggaccgatatcagtctgcacagcagcagcacccagccgtggccagggatgtcccagggagttcccatcatgagcagagttggcaaggtctcatggctgggaacgtggataaatgtgtggggtttgggtttcctgggggctcatcatgggaattcagcaagaaaagttggtgtgcaaccaatatagcctgaaaactcgagaacgtgtggctgttatccagaatgacccaaacacagtggtatgtacagtcagttgttagatgaaataggaaaaagaacacggcctttggtgttctccttccctcagcaaaacttcctcttggctgagtctacttgttaggcattttctagcttcagtttcacctcccttccccatctgagacactcctgttgctcacgtgcacttcttgtggcagtgccagcagcagtgcagctgctgctcatggccatcagcaccctggcactgtgagccccgggcttctcctggctccctgggcctcctcctccctcgctcccaccagggctgagcctgcagggagcggggctgcaagcaaagggctctgctggggggcaaagccagagcttctctgcctgccccggctgtagtttctgctaagtgaatagaggtgtcaatgaaattgtggggaaacacagcgagcaaagcaggaa

SEQ ID NO: 8 - DNA, 475 bp, Gallus(red jungle fowl)
atttcagtgttgagtgtattttccagagttagacacaccaaatctgactgccttgacttattcaaatgtgtgaggttgctggctgaaggccctgcatctacatcacatctacaaagcaaccaccgtgcagacgctccaacccttccgttcagccccggataacaacgcagtacgaaaagccggcaaacgtggcaggagagttttcctctcgaatcgatgcggctttgctttggctgaagttgtgcggggaaaaccgttccccgatctgcagcgttctgctgaataacgccccaaagttacagcgatgcgcacacgggaacggctctcgcagcgattccaggcgttgtccaaagagggaaagtctcaacacctcgagactccatctcaaacctatccaatgagacgcacgtgggaagagatgcggataaaacaacaataataataatagtaataaagcagagaagggggaaaaaaa

SEQ ID NO: 9 - DNA, 390 bp, Gallus(red jungle fowl)
ggcacgatgggctatttgtagctaacagataagcaaacgctgtctctctctccctatatattttgcttataccacagaaatggacgtcaaaaggtaaggaaataatacctgtttctcttaatttatttatttttctttgagaaaatgcatttgttaagctgcttttttttctgttccattccccatctccctctcctcccctacccaagggggggaaatagacagaaaagggtggagtgatgaaaaagggagaaatcacggctcaggttggacagctcatagtgaatgagaaaagccagaacctgcggcgtttgcaagctcagagaaatgagttgaacgctaaaggtaaagctcagttcctctcaccgtccttacctgggcctgtatgga